(19) United States
(12) Patent Application Publication
(10) Pub. No.: US 2022/0241512 A1
(43) Pub. Date: Aug. 4, 2022

(54) Apparatus for Detecting a Dose of Medicament Delivered from an Injection Device

(71) Applicant: Sanofi, Paris (FR)

(72) Inventor: Michael Helmer, Frankfurt am Main (DE)

(21) Appl. No.: 17/603,747

(22) PCT Filed: Apr. 16, 2020
(86) PCT No.: PCT/EP2020/060720

§ 371 Date: Oct. 14, 2021

(30) Foreign Application Priority Data
Apr. 18, 2019 (EP) 19305506.8

(51) Int. Cl. A61M 5/315; G06T 7/246

(52) U.S. Cl. CPC A61M 5/31568; A61M 5/3157; G06T 7/248; A61M 2205/3306; A61M 2005/3126; A61M 2205/3553; A61M 2205/3327; A61M 2205/581; A61M 2205/3592

(57) ABSTRACT

Disclosed is an apparatus for detecting a dose delivered from an injection device having a dose setting mechanism that allows different dosages to be delivered. The accessory has an attachment portion configured to attach the accessory to the injection device, and a body that houses an optical sensor arrangement of the injection device includes a cartridge assembly and a housing, the housing contains a dose dispensing mechanism and a dose setting mechanism, the dose dispensing mechanism includes a part that moves in response to movement of the dose setting mechanism and indicates a dose dialed by the dose setting mechanism. The housing further includes a window aligned with the part of the dose dispensing mechanism to allow the optical sensor arrangement to detect a location of the part that moves in response to movement of the dose setting mechanism to determine the dose dialed by the dose setting mechanism.

### CROSS REFERENCE TO RELATED APPLICATIONS

The present application is the national stage entry of International Patent Application No. PCT/EP2020/060720, filed on Apr. 16, 2020, and claims priority to Application No. EP 19305506.8, filed on Apr. 18, 2019, the disclosures of which are incorporated herein by reference.

### TECHNICAL FIELD

The present disclosure relates to an apparatus for detecting a dose of medicament delivered from an injection device having a dose setting mechanism that allows different dosages to be delivered from the injection device.

### BACKGROUND

A variety of diseases exists that require regular treatment by injection of a medicament. Typically, a medical practitioner formulates a dosage regime that manages the timing and dosage of the injections a patient should follow. Thus, the timing and/or the dosage of the injections can vary between patients and between injections. Often, as part of the dosage management regime, users are required to record parameters of the injections, for example to monitor effectiveness of the treatment or as feedback during the calculation of parameters for subsequent injections. This could be achieved through the keeping of a manual data logbook.

The injections can be performed either by medical personnel or by patients themselves by using injection devices. Injection devices (i.e., devices capable of delivering medicaments from a medication container) typically have a syringe connected to a medicament container and a dose dispensing mechanism for driving the medicament through the needle. The medicament chamber may be re-useable, wherein the dose dispensing mechanism is designed to be reset, allowing an empty medicament cartridge to be replaced by a new one. Alternatively, the injection device may be disposable, wherein, upon the contents of a pre-filled medicament container being emptied, the injection device is disposed of. In some examples, the injection device includes a dose setting mechanism that allows a user to set or ‘dial in’ an amount of medicament to be administered.

As an example, type-1 and type-2 diabetes can be treated by patients themselves by injection of insulin doses according to a dosage regime, for example injections once or several times per day. WO2004/078241 discloses a suitable injection device typically referred to as a pen, and references to pen herein are interchangeable with injection device. It is known for a disposable pen to be provided with a set of one-way needles that are attached to the pen before each use. The insulin dose to be injected and prescribed by the dosage regime can then, for instance, be manually selected through the dose setting mechanism by turning a dose knob to the required volume. The dose is then injected by inserting the needle into a suited skin portion and pressing an injection button of the dose dispensing mechanism. As part of the management of the dosage regime, the user records parameters of the injection. Such parameters, for instance, may be one or more of; the date and time of injection, blood sugar results, medication and dose, and/or diet and exercise information.

### SUMMARY

According to various aspects of the present specification, there is provided an accessory for an injection device, wherein the accessory optically monitors the displacement of a part of the injection device's dose dispensing mechanism. By monitoring the displacement of the dose dispensing mechanism's part, the accessory can detect a quantity of delivered medicament that is derived directly from the drive mechanism, and not from the displacement of a bung (also referred to as a stopper or plug) in the medicament cartridge. This takes advantage of the fact that there can be some disparity between the dose indicated as delivered by movement of the stopper and that indicated as delivered by movement of the drive mechanism because of flexing under force of components of the injection device and because of variation (within permitted tolerances) of the sizes and shapes of components in the drive mechanism and the cartridge. With a system in which displacement of the stopper is used to detect a quantity of delivered medicament, the determined quantity of medicament may not match the quantity that the user set into the device for delivery and the user may become concerned or even alarmed by a discrepancy between the dose that they thought they were administering and the dose that they were informed had been delivered. For instance, the user may become concerned that the intended dose had not been correctly set. The user could incorrectly believe that they have administered too much medicament. The user could incorrectly believe that they had administered too little medicament and decide erroneously to administer a top up dose.

In the exemplary embodiments, the accessory includes a body that houses an optical sensor arrangement. The body houses the optical sensor arrangement to view a scene through a window in the body and so that, in use, the optical sensor arrangement faces the part of the injection device on which the accessory is attached. The optical sensor arrangement faces a portion of the injection device wherein the part of the dose dispensing mechanism can be viewed. When the dose dispensing mechanism is provided adjacent the medicament chamber to act on a bung, displacement of the dose dispensing mechanism's part can be viewed through a window in the injection device without viewing through the medicament chamber. That is, by viewing the displacement through a window in the portion of the dose dispensing mechanism, the optical characteristics can be more readily controlled.

For example, the body houses the optical sensor arrangement so that a sensing surface of the optical sensor arrangement is arranged substantially parallel to the displacement movement of the part of the dose dispensing mechanism being monitored. Thus the optical sensor arrangement views the part orthogonal to the movement

In some examples, the optical sensor arrangement includes an image sensor. Here, the image sensor is arranged to capture an image. The image sensor may include a photodetector and a lens assembly providing suitable optical characteristics of the image sensor, for instance to provide a suitable field of view, focal distance, aberration control or the like. In an exemplary embodiment, the optical sensor arrangement has a field of view corresponding to a range of movement of a last dose nut (LDN) of the dose dispensing mechanism. Advantageously, because the accessory is specifically adapted to monitor the displacement of a part of the injection device's dose dispensing mechanism, for instance the LDN, the field of view of the optical sensor arrangement is able to be reduced as opposed to if the optical sensor arrangement was monitoring other parts of the injection device, for instance movement of a stopper of the medicament chamber, that typically has a greater range of movement.

In some examples, the optical sensor arrangement includes an optical emitter, such as a lighting unit. The lighting unit can be specified to improve the lighting conditions for the optical sensor arrangement. Here, the lighting unit is arranged to illuminate a portion of the injection device, for example a portion corresponding to at least the operational field of view of the optical sensor arrangement. That is, for example, the lighting unit is arranged to substantially illuminate the range of displacement movement of the part of the injection device's dose dispensing mechanism being monitored. In exemplary embodiments, wherein the displacement monitoring of the dose dispensing mechanism is monitoring the displacement movement of a last does nut (LDN), suitably, the optical emitter illuminates a portion of the optical sensor arrangement corresponding to a range of movement of the LDN. Similarly to the reduced field of view of the optical sensor arrangement as compared to monitoring movement of the bung, there is a reduced requirement on the lighting unit.

The accessory is adapted to measure a displacement of a part of the respective dose dispensing mechanism. In the exemplary embodiments, the measurement is calculated from a comparison of a first image recording the dose dispensing mechanism before an injection, that is, before the dose dispensing mechanism is operated to make an injection, as compared to a second image recording the dose dispensing mechanism after the injection. In exemplary embodiments, the images record a movement of said part of the dose dispensing mechanism relative to another part. Said part is suitably arranged to move in response to operation of a dose setting mechanism. For instance, said part may rotate relative to another part of the dose dispensing mechanism as the dose setting mechanism is activated. In examples such as these, the parts may include cooperating threads and said relative rotation causes one of the parts to advance along the corresponding thread. When the dose dispensing mechanism is activated, there is no relative movement between said part of the dose dispensing mechanism being monitored and the other part. In one exemplary embodiment, said part being monitored is a fill-level indicator and the other part a sleeve.

According to the exemplary embodiments and a further aspect, there is therefore provided an apparatus comprising the accessory, an injection device, and a processor for image processing. The processor may be integral to the accessory, for instance, housed in the body, or the processor may be remote from the accessory. In exemplary embodiments wherein the processor is remote to the accessory, the accessory includes a communication module for communicating the images to a remote device such as a smartphone, tablet computer, smartwatch or other independent device (e.g., laptop or PC). In some exemplary embodiments, the accessory includes a controller for controlling the optical sensor arrangement to complete various image acquisition steps. The controller may also control the communication module to transmit the acquired images to a remote device for image processing. Thus the main electronic processing is completed by the remote device, utilising the electronics of the remote device that does not then need to be replicated on the accessory. A power supply powers the optical sensor arrangement, controller or communication module as required. In some examples, the power supply is housed in the body of the accessory.

In the exemplary embodiments, a processor completes image processing steps to calculate a displacement of the part of the dose dispensing mechanism. The part is configured to move along a shaft portion of the dose-dispensing mechanism in response to operation of the dose setting mechanism. For instance, the part is engaged to the shaft portion via a cooperating thread and the part is advanced along the thread by rotation of the dose setting mechanism. When the dose dispensing mechanism is activated, the part is configured not to move relative to the shaft portion. In exemplary embodiments, the measurement is a displacement of a last dose nut (LDN) along a drive sleeve (DS). Here, the injection device is configured to move the LDN along the drive sleeve corresponding to movement of a dose setting mechanism. Typically the LDN has a restricted movement along the DS and reaches an end stop corresponding to a maximum dispensed volume (e.g., an empty cartridge) so that a dose cannot be dialed in to the dose setting mechanism that would exceed the remaining volume of medicament in the cartridge. After the dose dispensing mechanism is activated and the dose injected, the LDN remains in position relative to the DS before a further dose is set by the dose setting mechanism. The processor may calculate a displacement between the LDN and a reference on each image. The displacement is calculated as a difference in the position of the LDN between the images. Here, the processor can calculate a dose volume of the medicament that the dose dispensing mechanism is set to dispense. The calculation may comprise further factors such as characteristics of the DS screw pitch and diameter or associated characteristics of the dose dispensing mechanism.

For example, the processor calculates the displacement of the dose dispensing mechanism's part with respect to a reference. The reference may be a gauge formed on the window or a distinguishing mark on a body or other component of the injection device. However, the reference may be a mark or edge of another part of the dose dispensing mechanism. For instance in exemplary embodiments, the dispensing mechanism comprises a nut threaded to a shaft and the part being monitored is the nut and a portion of the shaft is the reference. In one embodiment, the nut is a last dose nut and the shaft is a drive sleeve. The processor may apply edge recognition algorithms to the processing to identify one or both of the reference edge and an edge of the part being monitored. Additionally or alternatively, one or both of the part being monitored or the reference may be provided (e.g., by material choice or by treatment) with a colour of high definition against the other parts in the viewing portion of the optical sensor arrangement.

For example, the processor calculates the displacement by evaluating the number of pixels in the images by comparing the number of pixels between the LDN and reference in a first image against the number of pixels between the LDN and reference in a second image after injection. The number of pixels may be counted in a linear line between the part and the reference. Here, the line may be along the axial direction of the injection device.

In exemplary embodiments, the processor calculates the displacement. The processer may convert the displacement into a dialed dose measurement. The processor may cause the dialed dose measurement to be transmitted to the accessory. Here, the accessory includes a communication module for receiving the dialed dose measurement. A controller controls the communication module and the controller may control the communication module to communicate the received dialed dose to a display. Here, a display is housed in the body of the accessory. The display is arranged to face the user and may be opposed to the optical sensor arrangement. A power supply is provided to power the display, controller and communication module. The display may also display other information received by the communications module, for instance the time or dose of a next injection. Additionally or alternatively, the processor may transmit the dialed dose measurement for storage in an electronic log book. For instance, the processor may communicate with an electronic log-book program or the like or the processor may process the images as part of an integrated electronic log-book program.

In some exemplary embodiments, the accessory comprises one or more switches. The switches may interact with a controller to initiate when to record an image and when to transmit an image via a communication module to a remote device. The switches may be manually operated to indicate events before and after an injection has occurred. Alternatively, one or more switches may be automatically activated to indicate one or more of the events in the injection process. For instance, a switch may be automatically activated to indicate the removal of a cap from the injection device or the operation of the dose dispensing mechanism or dose setting mechanism.

In the exemplary embodiments including a controller, the controller may include a memory to store one or more images received from the optical sensor arrangement.

In the exemplary embodiments including a communication module, the communications module may be a wireless communications module. For example, the wireless communication module is a short distance communication module. The method of operation may include the user completing a pairing step to pair the communication module to the remote device to establish a unidirectional or bidirectional mode of communication.

In the exemplary embodiments, the accessory includes an attachment portion. The attachment portion allows for the attachment of the accessory to the injection device. Here, the attachment portion restricts relative movement between the accessory and injection device in at least one direction. Advantageously, the restricted movement allows the accessory to be physically attached and held to the injection device, whilst also facilitating the location of the optical sensor arrangement in the required location. The attachment portion may either be separate from the accessory or integral to the body of the attachment portion. Thus, in some examples, the attachment portion is attachable and detachable to an injection device, which allows the attachment portion to be used on multiple injection devices. This is particularly useful when the multiple injection devices are disposable injection devices as the resources of the accessory can be reused across multiple devices. In some examples, the attachment portion is formed as an integral part of the injection device, and the body of the accessory may be the body of the injection device. An integral arrangement might be useful for a reusable injection device.

An axial direction is a direction along an axis of the injection device, for instance, coaxial with an axis of a syringe or the direction of movement of the medicament chamber's bung. The attachment portion may be arranged to engage the injection device by attachment through relative movement between the accessory and respective injection device in the axial direction, and herein termed the engagement movement. The engagement movement may be in the axial direction in a proximal-distal direction or the reverse distal-proximal direction. Here, the proximal-distal direction is from the dose dispensing mechanism towards the cartridge and the distal-proximal direction is the opposite, from the cartridge towards the dose dispensing mechanism. In the exemplary embodiments, the attachment portion restricts relative movement between the accessory and the injection device in a direction opposed to the engagement movement. In the exemplary embodiments, abutment between the attachment portion and injection device provides the restriction to the movement.

In embodiments comprising a linear engagement movement in one of the axial directions, the attachment portion and injection device may be arranged to engage at a tapered portion. Here, at least one of the respective parts is tapered so that the parts are cooperatively engaged by friction. In one embodiment, the attachment portion is tapered. Here, the tapered portion of the attachment portion includes a first area forming opposed points spaced around the pen, and a second area having opposed points spaced around the pen, wherein the distance between the points of the first area is smaller than the distance between the points of the second area. Thus the restricted movement is provided by friction generated by a force applied by the engagement movement. Additionally or alternatively, the injection device is tapered at a connection area adapted to receive the accessory.

In additional or alternative embodiments comprising a linear engagement movement, the attachment portion and injection device may be arranged to provide a positive location. For instance, one of the injection device or the attachment portion comprises a resilient portion over which the other part is arranged to pass. Here the resilient portion provides a localised restriction to a separation distance between the parts. Pushing the two parts together by the engagement movement and so that the respective part moves over the resilient portion produces a positive location that provides feedback to the user that the attachment of the accessory has been completed. It also provides initial resistance to the removal of the accessory in the reverse, disengagement movement direction.

In some embodiments, the attachment portion may include a locator to locate the accessory on the injection device. The locator may provide a rotation key to align the accessory and injection pen in a rotational alignment relative to the axial direction, and the key may prevent rotational movement of the accessory relative to the injection device when attached via a linear engagement movement along the axial direction. Here, the attachment portion and injection device are arranged to have cooperating alignment features. The cooperating alignment features may comprise a non-symmetrical cross section relative to the axial direction or may comprise a protrusion and recess on respective parts. The locator aids the alignment of the optical sensor arrangement and part of the dose dispensing mechanism being monitored.

The engagement movement may include a relative movement between the accessory and injection device in an additional or alternative direction. For instance, the engagement movement may include a rotational movement about the axial direction. Thus the attachment portion may engage the injection device through a twisting motion, wherein the attachment portion and injection device include respective cooperating features. Here, the, cooperating features are caused to cooperate during the engagement movement wherein the attachment portion restricts relative movement in both the distal-proximal and proximal-distal axial directions. The cooperating features may also engage to restrict rotational movement in one direction. For instance, the attachment portion and injection device may comprise cooperating threads or cooperating bayonet features or the like.

A radial direction is a direction transverse a direction along an axis of the injection device, for instance coaxial with an axis of a syringe or the direction of movement of the medicament chamber's bung (that is transverse the axial direction). The attachment portion may be arranged to engage the injection device by attachment through relative movement between the accessory and respective injection device in the radial direction, and herein termed the engagement movement. For instance, the attachment portion and injection device may comprise cooperating pairs of recesses and protrusions that act as a push button fit. Or alternatively, the attachment portion may be arranged to partially enclose the injection device, wherein the attachment portion is resilient and acts as a clip around the injection device. Here, further alignment features may be provided to aid rotational alignment of the optical sensor arrangement and part being monitored.

In the exemplary embodiments, the attachment portion engages with the injection device at a mid-point of the injection device. Thus, when attached, the injection device extends from either side of the accessories body. This allows a cap to be placed and removed from the injection device without requiring removal of the accessory. Thus the accessory can remain attached to the injection device during operation of the injection device.

The body of the accessory may include an attachment portion and an operative portion. Here, the operative portion houses the optical sensor arrangement and the optional display. The operative portion may extend away from the attachment portion in an axial direction of the injection device. The operative portion may only cover a portion of the surface of the injection device about a radial surface. Thus the accessory can be attached without covering the entirety of an information label secured about the injection device.

In one exemplary embodiment, the attachment portion comprises a ring. The ring is adapted to fit and encircle the injection device.

In exemplary embodiments, the accessory is adapted to attach to an injection device being a pen type device. The pen includes a medicament chamber, a dose dispensing mechanism, and a dose setting mechanism. In some examples, the dose dispensing mechanism and dose setting mechanism are assembled in a housing. Here, the housing may provide a connection to attach the medicament chamber, or the medicament chamber may also be assembled within the housing. A window is formed in the housing. The window is located relative to the dose dispensing mechanism and so as to expose a part of the dose dispensing mechanism being monitored. The window is optically transmissive to allow the optical sensor arrangement to monitor the respective part. In some embodiments, the window is a recess formed through the housing. Here the window may be left open or may be closed by an optically transmissive part. Alternatively, at least an area of the housing is formed to be optically transmissive, for example the housing is formed from an optically transmissive material. In some exemplary embodiments, the housing is covered by an information label. The information label may include a removable section to expose the window. For instance, the label may be formed with an area defined by perforations, and the user may remove the area defined by the perforations prior to attaching the accessory.

In the exemplary embodiments, the injection device includes a housing and the housing provides a receiving portion to receive an attachment portion of the accessory. The receiving portion cooperates with the attachment portion to locate and secure the accessory to the injection device. The window may be located adjacent or spaced from the attachment portion. The attachment portion may include a locator to correspond to a locator of the accessory to aid alignment of the accessory to the window. In some examples, the receiving portion may be provided adjacent the medicament chamber or connection between the housing and medicament chamber.

According to an exemplary aspect, there is therefore provided an apparatus comprising an accessory and an injection device 200. The accessory includes a body that houses an optical sensor arrangement. The body defines an attachment portion for attaching the accessory to the injection device. The injection device includes a cartridge assembly assembled to a housing. The housing contains a dose dispensing mechanism and a dose setting mechanism. The dose dispensing mechanism includes a part that moves relative to a reference in response to movement of the dose setting mechanism. The housing includes a window aligned with said part of the dose dispensing mechanism. Wherein the attachment portion is specifically adapted to align the optical sensor arrangement to the window. The accessory and injection device may be provided as a kit of parts or individually for use with each other.

According to a further aspect there is provided a method of managing a dosage regime. The method comprises optically monitoring a displacement of a part of an injection device and electronically logging the result as part of a dosage management regime. The method further comprises attaching an accessory of the previous aspects to the injection device to form an apparatus of previous aspects.

The method may comprise operatively connecting an attachment portion of the accessory to a receiving portion of the injection device. The step of operatively connecting the attachment portion and receiving portion comprises moving the attachment portion relatively to the injection device. The step of operatively connecting the accessory aligns an optical sensor arrangement to a viewing window in the injection device such that the optical sensor arrangement can view a movement of the part of the dose dispensing mechanism being monitored. The step of operatively connecting the attachment may comprise aligning and engaging a key on the accessory to a key on the injection device in order to rotationally align the optical sensor arrangement and window about an axis of the injection device.

In some examples, the method comprises steps of removing and replacing a cap on to the injection device before and after an injection. In such examples, the arrangement of the attachment portion facilitates the removal and replacement of the cap whilst the accessory remains attached to the injection device.

Prior to the attachment of the accessory to the injection device, the method may comprise removing an area of an information label covering a housing of the injection device. The label removal step may comprise removing a portion of the label. For instance, by tearing through perforations provided in the label to reveal the window.

The method may comprise a pairing step to pair the accessory with a remote device. For instance, to pair a communications module in the accessory with a corresponding module in the remote device. The pairing may be activated by operating a switch of the accessory or the like.

In the exemplary method, a pre-injection image acquisition step comprises operating the optical sensor arrangement to capture an image of the position of a part of the dose dispensing mechanism. And a post-injection image acquisition step comprises operating the optical sensor arrangement to capture a second image of the position of a part of the dose dispensing mechanism. In some examples, the image captures the position of the part relative to a reference. The pre-injection and post-injection image acquisition steps may be activated by operating a switch of the accessory. The switches may be operated manually or automatically upon another event. The image acquisition step may also comprise operating a lighting unit to illuminate the viewing portion. The acquisition steps may comprise causing a communications module to transmit the images to a remote device. The images may be transmitted after completion of the injection or at stages throughout the injection.

In the exemplary embodiments, the method comprises post processing the images to calculate a displacement of the part of the dose dispensing mechanism. The post processing may be completed by a processor of a remote device. The processing may comprise the steps of calculating a number of pixels between a reference and the part in the pre-injection image, and calculating a number of pixels between a reference and the part in the post-injection image. The processing step may comprise comparing the difference in number of pixels between the images to determine the displacement of the part. The number of pixels may define a line along the axis of the injection device and defining a line from edge centre points of the reference and part. The processing step may also comprise converting the displacement into a dose-dialed measurement.

In an embodiment, the processor for the processing step is part of a remote device. Here, the exemplary embodiments include a step of transmitting the pre and post injection images to the processor, where the transmission may be completed by a communications module of the accessory. Here, the communications module may also be paired to the remote device to receive transmissions. For instance, the accessory may include a display wherein the method comprises controlling the accessory to operate the display to display information received from the remote device.

In exemplary embodiments, the method comprises a step of logging the dose dialed calculation as part of an electronic log-book to record the dose dialed measurement. The electronic log book may also log details of the injection such as the time of the injection based on the time of receipt by the processor of the one or other of the images.

According to the exemplary embodiments, the method comprises repeating the steps of logging a dose dialed measurement of subsequent injections. The method further comprises removing the accessory from a first injection device and reattaching the accessory to a second injection device. For instance, when a first injection device is emptied, the accessory is removed and replaced on a second, replacement injection device with medicament remaining therein.

According to the exemplary embodiments, there is therefore provided an improved accessory for an injection device, apparatus comprising the accessory and one or more injection devices, and a method of managing a dosage regime as set forth in the appended claims. Other features of the present disclosure will become apparent from the description and elsewhere in the application. By using an accessory to monitor the movement of a part of the dose dispensing mechanism, a dose dialed measurement can be electronically recorded as part of a dose management method. Moreover, the accessory can convert injection devices to be capable of electronic capturing of does dialed measurements with limited changes to the injection device. By monitoring a part of the dose dispensing mechanism, it is possible to provide controlled viewing characteristics. Furthermore, by monitoring the LDN of the dose dispensing mechanism, a field of view of the optical sensor arrangement is smaller than a stroke length of the bung in the medicament chamber.

### BRIEF DESCRIPTION OF THE FIGURES

Exemplary embodiments are described with reference to the accompanying drawings, in which:

FIG. 1 shows a schematic view of an injection device;

FIG. 2 shows an exploded parts view of the injection device of FIG. 1;

FIG. 3 shows a partial cross-sectional view through a grip end of the assembled injection device shown in FIG. 2;

FIGS. 4 and 5 show perspective views of alternative injection devices;

FIG. 6 shows a perspective view of an accessory for an injection device;

FIG. 7 shows a perspective view of an apparatus comprising an injection pen and an accessory attached thereto;

FIG. 8 shows a partial perspective view of FIG. 5 showing a part cut-away detail;

FIG. 9 illustrates a movement of a last dose nut along a drive sleeve of the injection device shown in FIGS. 1-3;

FIG. 10 details a schematic plan of a control layout; and

FIG. 11 shows a method of managing a dosage regime.

Reference will now be made in detail to the embodiments of the present disclosure, examples of which are illustrated in the accompanying drawings, wherein like reference numerals refer to like elements throughout.

### DETAILED DESCRIPTION

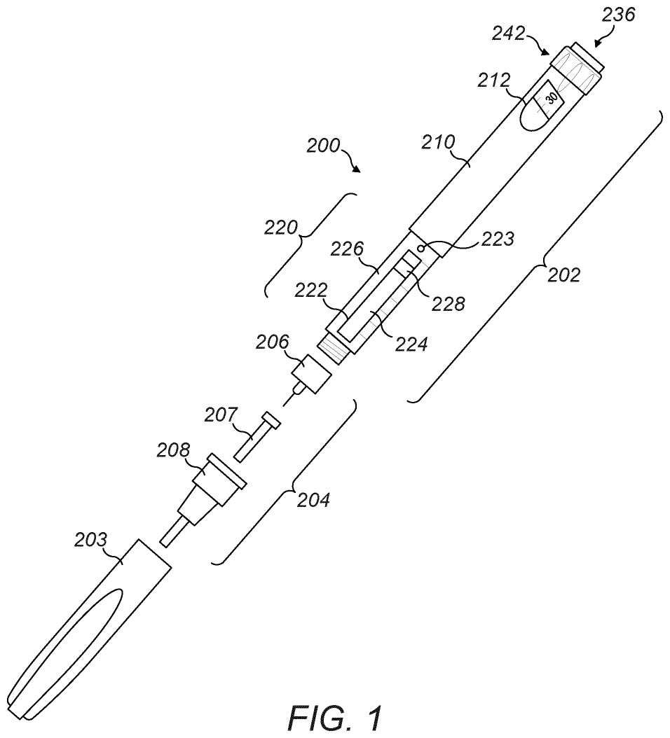

FIG. 1 is an exploded view of an injection device 200 suitable for use with exemplary embodiments. The injection device shown is often referred to as an injection pen or pen. Various design of pen are known and whilst a brief description is given herein, it will be appreciated that the specific construction of the pen may alter and vary from the following description.

The injection device 200 has a distal end and a proximal end. The term “distal” refers to a location that is relatively closer to a site of injection, and the term “proximal” refers to a location that is relatively further away from the injection site.

The injection device 200 comprises a grip assembly 202, a cap 203 and a needle assembly 204. The grip assembly is formed from a housing 210 and a cartridge assembly 220. The cartridge assembly 220 includes a cartridge holder 222 for containing a cartridge 224 containing medicament. As shown, housing 210 is substantially cylindrical and has a substantially constant diameter along its longitudinal axis from a proximal end to a distal end. The longitudinal axis has a proximal-distal direction that extends from the proximal end to the distal end and the reverse distal-proximal direction. A label 211 is provided on the housing 210. The label includes information about the medicament included within the injection device 200, including information identifying the medicament. The information identifying the medicament may be in the form of text. The information identifying the medicament may also be in the form of a colour. The information identifying the medicament may also be encoded into a barcode, QR code or the like. The information identifying the medicament may also be in the form of a black and white pattern, a colour pattern or shading.

The cartridge assembly 220 is assembled to the housing 210 to form the grip assembly 202. In some examples, the proximal end of the cartridge assembly 220 includes a connection part and the distal end of the housing 210 includes a corresponding connection part that cooperatively engage with each other to connect the two parts. As shown, the cartridge holder 222 is substantially cylindrical with a hollow receiving for the cartridge 224. The cartridge includes a stopper 228 that can be advanced within the cartridge 224 during use to expel medicament from the cartridge. Here, it will be appreciated that the needle assembly 204 cooperates with the grip assembly to serve as a conduit for the medicament during injection.

The cartridge holder 222 has a porthole 226 in a side thereof. The porthole 226 allows the user to view the cartridge 224 through the porthole 226 when the cartridge 224 is contained in the cartridge holder 222. FIG. 1 shows a stopper 228 of the cartridge 224 visible through the porthole 226. FIG. 1 shows the cartridge holder 222 having one porthole 226 however, the cartridge holder 222 may instead have more than one window 107. For example, the cartridge holder 222 may have a first porthole 226 located on one side of the cartridge holder 222 and a second porthole located on a second, in some cases opposing, side of the cartridge holder 222. Thus a first side of the cartridge 224 within the cartridge holder 222 may be visible through the first porthole 226 while a second, different side of the cartridge 224 may be visible through the second porthole. Other porthole configurations may be used.

The needle assembly is shown comprising a needle 206, an inner needle cap 207 and an outer needle cap 208. A needle 206 of the needle assembly 204 can be affixed to the cartridge holder 104 such that the needle 106 is in fluid communication with the medicament in the cartridge 224. The needle is protected by the inner needle cap 207 and the outer needle cap 208,

The removable cap 203 attaches to the cartridge assembly. The cap 203 at least partially covers the cartridge holder 222, and hence cartridge 224, when attached to the grip assembly. The cap 203 may also be attached to the grip assembly such that it at least partially covers the cartridge holder 222 with or without one or more of the needle 106, inner needle cap 108 or outer needle cap 110 being present.

The cartridge holder 222 may have a cap retaining feature 223 on an outer surface, for example adjacent a proximal end of the cartridge holder 222, and adjacent the attachment to the housing 210. Thus the cap 203 may substantially cover the cartridge assembly when fitted. The cap retaining feature 223 engages with a corresponding coupling feature on an inner surface of the cap 203 to hold the cap 203 in place when attached to the grip assembly. The cap retaining feature 223 may comprise one or more of a ridge, groove, bump, lock, and/or pip. In some examples, the cap retaining feature is located on the housing 210 of the injection device 200.

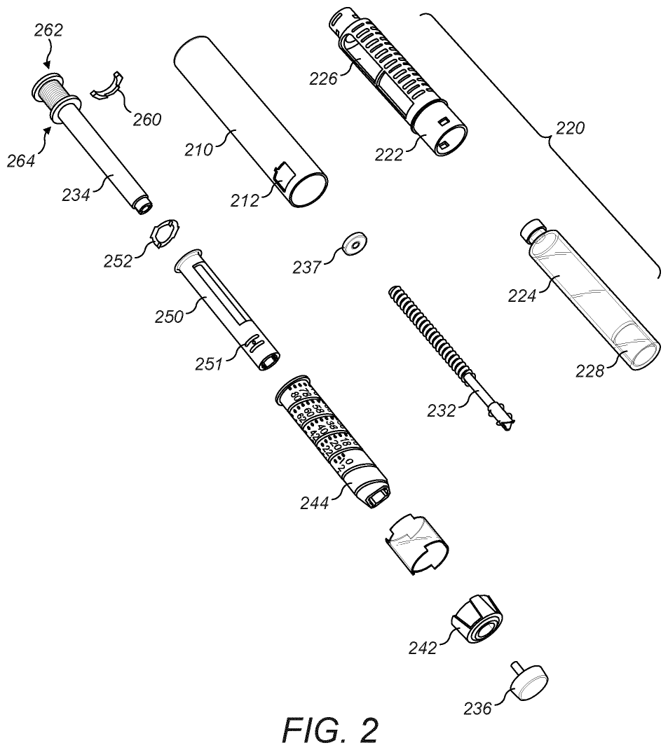

As shown in FIG. 2, the housing 210 houses a dose dispensing mechanism and a dose selection mechanism. The dose setting mechanism is used to select a dose to be injected and the dose dispensing mechanism is activated to inject the dose. In this instance, the dose dispensing mechanism is activated to drive the stopper towards the distal end of the cartridge 224. The injection device 200 may be used for several injection processes until either the cartridge is empty or the expiration date of injection device 200 (e.g., 28 days after the first use) is reached. Injection device 200 may be single-use or reusable.

To drive the stopper 228 into the cartridge 224, the dose dispensing mechanism includes a piston rod 232, a drive sleeve 234, and a trigger button 236, which act together to drive a pressure plate 237 against the stopper 228 and into the cartridge 224. A medicament or drug dose to be ejected from the drug delivery device 200 is selected by turning a dosage knob 242, which is connected by a threaded insert 243 to a dose dial sleeve 244, where rotation of the dose dial sleeve 244 by the dosage knob 242 causes the selected dose to be displayed in a dosage window 212 in the housing 210 and causes a clicker 250 to interact with the drive sleeve 234 via a spring clutch 252. Together, the dosage knob 203, dose dial sleeve 230, and clicker 250 are a dose setting mechanism. The dose dial sleeve 244 is arranged around the clicker 250, which includes a feedback mechanism 251 that generates a tactile or audible feedback with rotation of the dose dial sleeve 244. The clicker 250 is coupled to the drive sleeve 234 with a metal clutch spring 252.

A last dose nut 260 (LDN) is provided on the drive sleeve 234. The last dose nut 260 advances with each dose dispensing operation to track the total medicament remaining in the cartridge 224. The trigger button 236 is depressed to activate a dose dispensing operation of the drug delivery device 100. The drive sleeve 234 includes flanges 262 and 264 that project from the drive sleeve. For instance the flanges may be radial flanges. The LDN 260 is a threaded part and, in some examples, may be a half nut. The drive sleeve includes a threaded bolt section that typically extends between the two flanges. As the drive sleeve is rotated by corresponding rotation of the dose setting mechanism, the LDN 260 is caused to move along the drive sleeve by cooperation of the respective threads. The LDN can be arranged to move from flange 162, which is a minimum flange indicating the starting position of the LDN when the LDN abuts the flange and the cartridge is full. The LDN iteratively moves along the LDN as each dose is injected. The LDN abuts the other flange, which is a maximum flange that prevents the LDN from moving and consequently prevents the dose dialed mechanism from dialing in a dose that would exceed the dose remaining in the cartridge.

While the dose setting mechanism is illustrated as the dosage knob 242, dose dial sleeve 244, and the clicker 250, as described above, one skilled in the art will appreciate that any number of different dose setting mechanisms are routine in the art for the purposes of setting a dose of a drug delivery device and aspects of the present disclosure are compatible with other such dose setting mechanisms. Similarly, while the dose dispensing mechanism is illustrated as including the piston rod 232, drive sleeve 234, trigger button 236, one skilled in the art will appreciate that a number of different dose dispensing mechanisms (e.g., drive mechanisms) are known in the art for the purposes of delivering or dispensing a dose of a drug delivery device and aspects of the present disclosure are compatible with other such dose dispensing mechanisms.

Continuing with the operation of the drug delivery device 200, turning the dosage knob 236 causes a mechanical click sound to provide acoustical feedback to a user by rotating the dose dial sleeve 244 with respect to the clicker 250. The numbers displayed in the dosage display 212 are printed on the dose dial sleeve 244 that is contained in the housing 210 and mechanically interacts with the drive sleeve 234 via the metal spring clutch 252. When the injection button 236 is pushed, the drug dose displayed in the display 212 will be ejected from the drug delivery device 100. During a dose setting operation, the drive sleeve 234 is helically rotated with the dose dial sleeve 234 spiraling outwardly in the distal-proximal direction. When the injection button 236 is pushed, the drive sleeve 234 is released and advanced distally, which causes rotation of the piston rod 232. The rotation of the piston rod 232 drives the pressure plate 237 against the stopper 228 of the cartridge 224, which drives the stopper 228 into the cartridge 224 to expel the medicament from the cartridge 224. A more detailed description of a representative drug delivery device is described in U.S. Pat. No. 7,935,088 B2, issued 3 May, 2011.

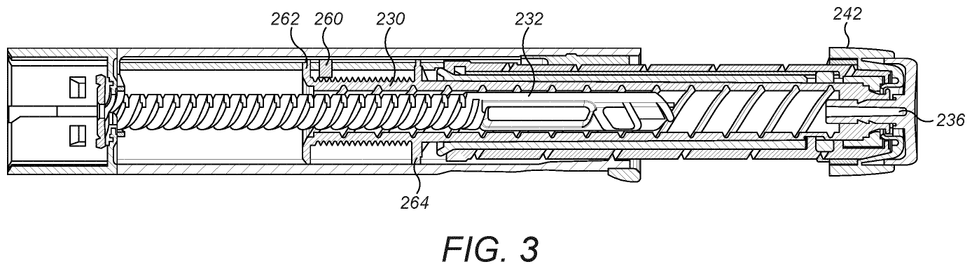

FIG. 3 shows the drug delivery device 100 at the end of a dose setting operation and prior to a dose dispensing operation, where the dose dial sleeve 244 and the drive sleeve 234 have been helically rotated with respect to the housing 210 and a threaded end 233 of the piston rod 232 to set the dose. The last dose nut 260 is shown advanced along the drive sleeve 234 from an initial position to a position indicative of the dose remaining in the drug delivery device 100. Upon dose dispensing of the injection button 236, the drive sleeve 234 advances into the housing 210 and a bearing nut 214 induces rotation of the piston rod 232. The bearing nut 214 sits fixed inside the housing 210 and has a threaded engagement with the piston rod 232. As the piston rod 232 rotates, the piston rod 232 is screwed forward (relative to the housing 210) because the bearing nut 214 cannot move. The rotation of the piston rod 232 drives the piston rod 232 and the pressure plate 237 proximally in the proximal-distal direction to drive the stopper 228 into the cartridge 224. Once dispensed, the drive sleeve is in a non-dose dialed position.

A medicament dose to be ejected from injection device 100 can be selected by turning the dosage knob 242, and the selected dose is then displayed via dosage window 212, for instance in multiples of International Units (IU). An example of a selected dose displayed in dosage window 12 may be ‘30’ IUs, as shown in FIG. 1. It should be noted that the selected dose may equally well be displayed differently, for instance using an electronic display.

Turning the dosage knob 242 causes a mechanical click sound to provide acoustic feedback to a user. The numbers displayed in dosage window 212 are printed on the sleeve 244 that is contained in housing 210. When needle 206 is stuck into a skin portion of a patient, and then injection button 236 is pushed, the medicament dose displayed in display window 212 is ejected from injection device 100. When the needle 206 of injection device 100 remains for a certain time in the skin portion after the injection button 236 is pushed, a high percentage of the dose is actually injected into the patient's body. Ejection of the medicament dose also causes a mechanical click sound, which is however different from the sounds produced when using dosage knob.

Whilst a pen injection device is briefly described in the present disclosure, other injection devices are envisaged, as is known in the art.

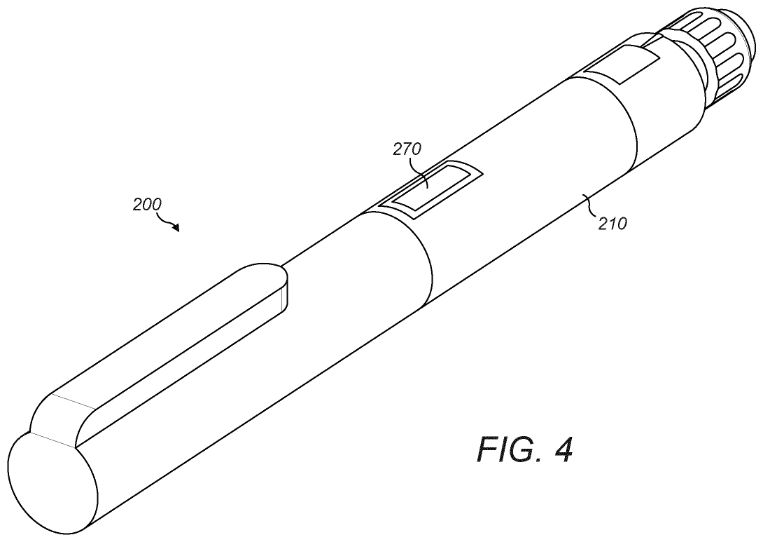

In exemplary embodiments and referring to FIG. 4, a window 270 is provided in the housing 210. The window is formed at the distal end of the housing 210. The window is formed in a portion corresponding to a portion of housing that accommodates a part of the dose dispensing mechanism the movement of which is to be monitored. The window is shown as being adjacent the connection connecting the housing 210 and cartridge assembly 220. The window 270 may be spaced from the proximal end of the housing so that the window is bounded by the housing 210, or the window may be arranged coincident with the proximal end so that one side of the window is open.

The window 270 is arranged to be optically transmissive such that an optical sensor arrangement can view through the window 270 to monitor displacement of the part of the dose dispensing mechanism being monitored. Thus the window 270 may be an open aperture through the housing. Alternatively, the window 270 may be an aperture through the housing that is filled with an optically transmissive part. For instance, an aperture filled with a pane. The window is sized to correspond to the extent of movement of the part being monitored. In the embodiment wherein the housing is cylindrical, the window may extend about a part of the circumferential face of the housing 210. For instance, the window 270 may subtend an arc of the circumferential face. The arc may subtend an angle greater than 10° or greater than 20° or greater than 30° or greater than 45°. The arc may subtend an angle less than 120° or less than 110° or less than 100° or less than 90°. When monitoring the displacement of an LDN, in the axial direction of the housing 210, the window 270 may extend substantially between the location of the minimum flange 262 and maximum flange 264 of the drive sleeve 234 when the drive sleeve 234 is in the non-dose dialed position

In one alternative embodiment, the window 270 is created by forming the housing 210 or the proximal end of the housing from an optically transmissive material. Here, the window 270 would extend around the full circumferential face of the housing 210. The window would extend in the axial direction in at least the corresponding extent of movement of the part being monitored. That is, when monitoring the movement of the LDN 260, between the minimum and maximum flanges 262, 264.

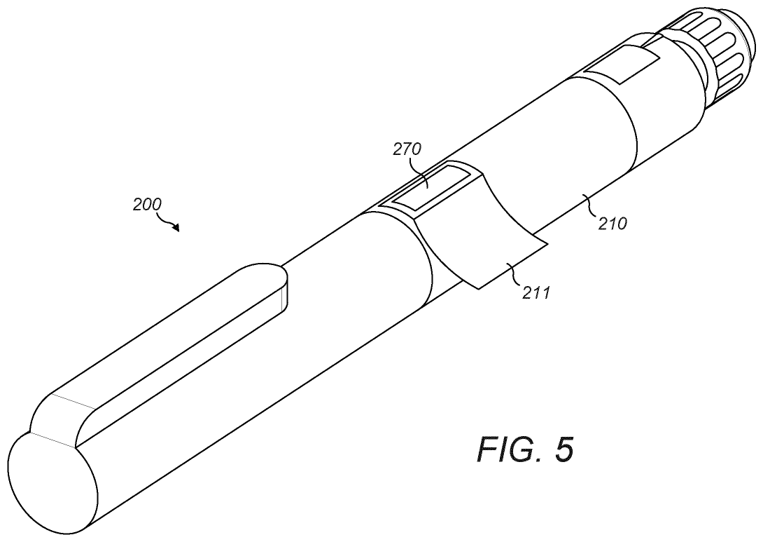

In FIG. 4, the label 211 affixed to the housing is shown as not being formed on the window 270. Referring to FIG. 5, the label may be affixed to the housing to cover the window and the area of the label covering the window removed before attaching an accessory to digitally monitor the displacement. Here, the label is provided with perforations along which the label can be torn or cut or folded to be removed from the area of the window.

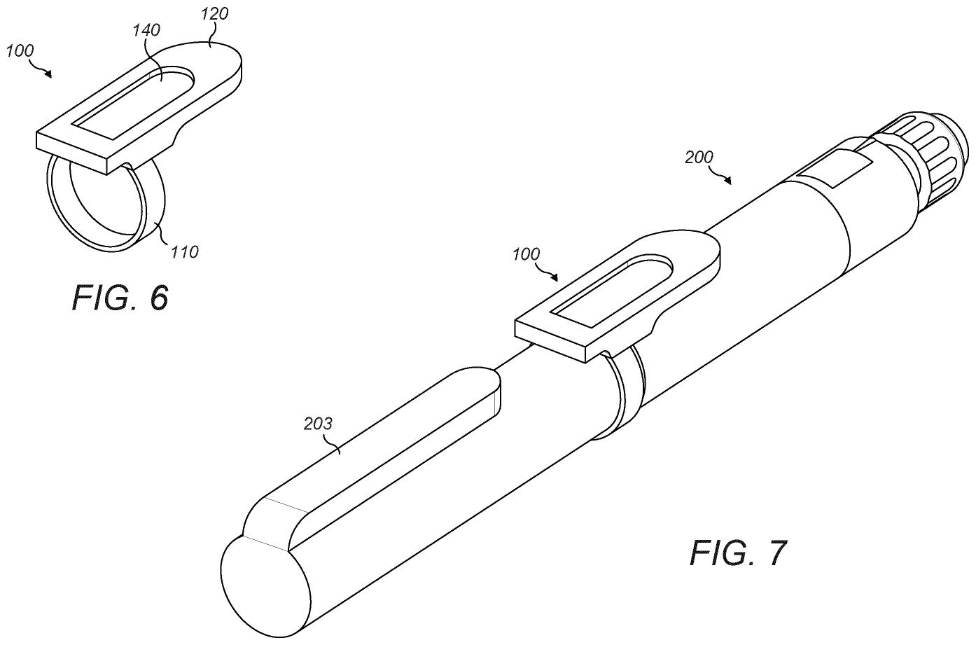

Referring to FIG. 6, an accessory 100 is shown. The accessory is adapted to be attached to the injection device 200. The accessory 100 includes a body defining an attachment portion 110 and an operative portion 120.

The attachment portion is arranged to allow the accessory to be attached to the injection device 200. The attachment portion 110 is shown in FIG. 6 as a collar that is adapted to locate around the injection device 200. The collar is a substantially enclosed ring having an internal diameter. Here, the collar is sized so as to have an internal diameter suitable to fit over the distal end of the cartridge assembly. That is greater than a size if the distal end of the cartridge. The accessory 100 can therefore be attached to the injection device 200 by relative lateral movement of the accessory and injection device along the longitudinal axis of the injection pen and in a distal-proximal direction. The accessory engages the injection device by friction or alternatively by a clipping or resilient deformation of respective parts on the accessory or injection pen.

In FIG. 7, the attachment 100 is shown attached to the injection device 200. The attachment portion 110 is shown engaged with a receiving portion of the injection device. The receiving portion is shown as being adjacent the connection between the cartridge assembly and the housing 210. The receiving portion may be a portion of the cartridge, for instance a proximal portion at a proximal end, or a portion of the housing, for instance a distal portion at a distal end. In exemplary embodiments, the receiving portion is not covered by the cap 203 when the cap 203 is attached. In FIGS. 6 and 7 the accessory is shown as engaged with the injection device by a friction fit between the attachment portion and the receiving portion. For instance, by a suitable sizing and tapering of the inside of the attachment portion and the exterior of the receiving portion.

The operative portion extends away from the attachment portion and along the axis of the intended injection pen. The operative portion 120 houses an optical sensor arrangement 130. The optical sensor arrangement 130 is an electronic device for monitoring the displacement of the part of the dose dispensing mechanism. For instance, by recording a digital picture of the part's position at two or more stages of the injection process. Here the optical sensor arrangement includes an image sensor 132, for instance an array of photodetectors such as a CCD or CMOS detector, that converts an optical image into a digital signal as part of an image acquisition process and as is known in the art. The array of photodetectors may be a one-dimensional array, for instance an array along the longitudinal axis, or the array may be a two-dimensional array in both the circumferential direction and the longitudinal axis. A lighting unit 134 may be provided as part of the optical sensor arrangement 130 to provide consistent lighting characteristics for the image acquisition, for instance one or more light emitters such as an LED array, and as is known in the art.

The optical sensor arrangement 130 is arranged in the operative portion 120 so as to face the window 270 of an injection device when attached thereto. The attachment portion extends away from the operative portion to enclose the injection device and the optical sensor arrangement is arranged to be directed in the same direction. For instance, if the attachment portion 110 extends from an underside of the operative portion 120, the optical sensor arrangement is arranged on the underside of the operative portion. Here, the underside of the operative portion may include an aperture through which the optical sensor arrangement 130 is directed. The aperture may be open or filled with optically transmissive material.

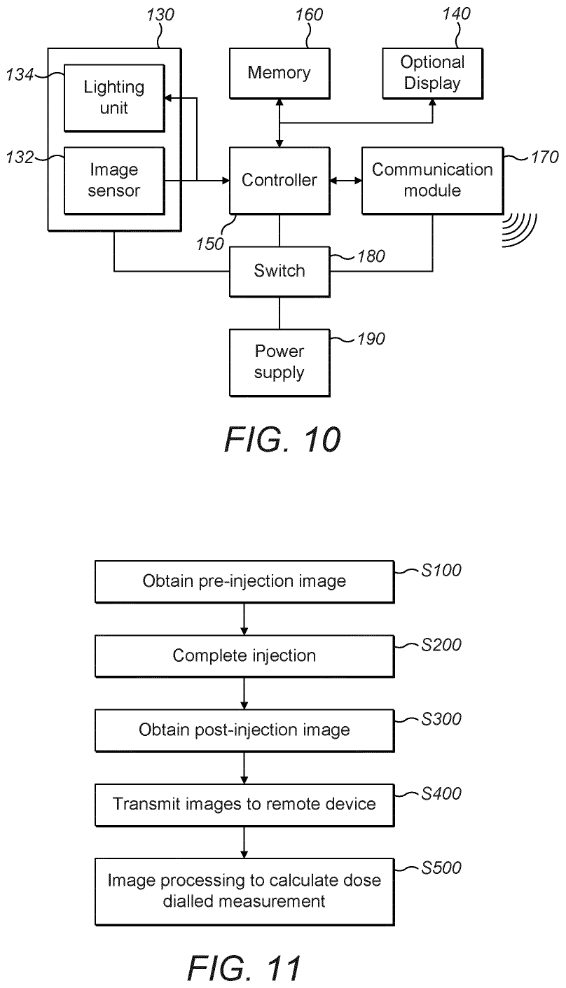

An optional display 140 is housed in the body of the accessory, for instance on an upper side (or user facing side) of the operative portion. The display 140 may be an e-ink display module, which require power to change the display but that are capable of displaying images (such as text) for periods without power. As will be described in relation to FIG. 10, the body may house further electronic modules such as a controller, power supply, communications module, and memory. The body may also provide one or more switches that can be manually operated to control one or more of the image acquisition and processing functions.

Referring to FIG. 8, the accessory 100 is aligned with the window 270 in the injection device, which is correspondingly aligned with the part of the dose dispensing mechanism being monitored. As explained herein, the part of the dose dispensing mechanism being monitored is a part that moves relative to another part to be indicative of a dose dialed in by the dose setting mechanism. In the exemplary embodiments, the part being monitored is the LDN 260 and its relative movement along the sleeve 234. As explained above, the LDN 260 moves between the minimum flange 262 and maximum flange 264 as doses are dialed in to the dose setting mechanism. When the dose dispensing mechanism is triggered, for instance by pressing the trigger 236, the LDN does not move along the sleeve. Rather, it remains static relative to the sleeve during dispensing. Thus the part moves along the sleeve during dose setting and then translates with the sleeve as the sleeve translates to drive the stopper into the medicament cartridge. Thus, the part moves in stages towards the maximum flange, with each stage representing a dispensing operation. When reaching the maximum flange 264, the LDN prevents further doses being dialed into the dose setting mechanism. The maximum flange indicates the LDN has moved along the sleeve indicative of the maximum medicament in the cartridge (i.e., the cartridge is empty, or would be empty if the dose was dispensed). Because the LDN indicates a level of medicament remaining in the medicament cartridge, or that would remain if the dialed in dose set by the dose setting mechanism were injected, the LDN may also be referred to as a fill-level indicator.

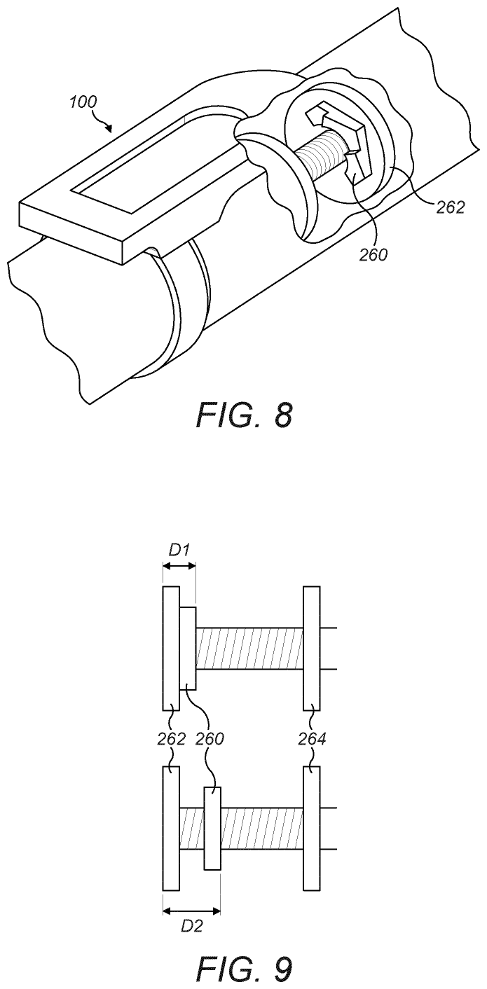

Whilst in the described embodiments, the sleeve moves axially in the distal-proximal direction when a dose is dialed into the dose setting mechanism, after injection, the sleeve returns to the same position in the housing. Thus the window can be arranged to be aligned with this position and the respective images recorded when the sleeve is in the non-dose dialed position. The dose dialed measurement can be calculated as a displacement of the LDN along the sleeve. As described above, the part has moved along the sleeve due to operation of the dose setting mechanism. Typically, the LDN part is rotated relative to the sleeve and the movement is driven by cooperating threads between the part and sleeve. Because the part has moved along the sleeve due to operation of the dose setting mechanism, an image before injection can be compared to an image after injection to calculate the displacement of the LDN. As shown in FIG. 9, the position of the LDN 260 in each image can be calculated from a reference. The reference may be a gauge formed on the injection device, such as a mark or edge of the sleeve. For instance, the reference may be an edge of the minimum or maximum flange 262, 264.

As shown in FIG. 9, the upper image represents a pre-injection image and the lower image represents a post-injection image and the dose dialed measurement is calculated by identifying the displacement of the LDN 260 by taking away the distance between the LDN 260 and reference after injection from the same measurement before injection. An image processing step calculates the measurement. For instance, the image processing step calculates the displacement by reference to the number of pixels between the reference and LDN 260 in each image. Here an edge detection algorithm may be applied to determine the respective edges. The components whose edges are detected may be coloured differently to other components in order to assist the edge detection. For instance, the LDN 260 and the flanges may be a dark colour compared to the other components, or vice versa. As long as there is sufficiently high contrast between the colour of a component and a colour of components adjacent to it and behind it (relative to the sensor), the edge can be reliably detected and its position can be reliably measured.

To obtain the correct alignment of the accessory and injection device so as to align the optical sensor arrangement 130 and window 270, cooperating locators may be provided to ensure rotational alignment of the respective accessory and injection device.

As shown in FIG. 10, a controller 150 controls the image acquisition steps of the image sensor 130. Memory 160 may be provided as required. The controller controls the optional display to display an image. Whilst the accessory may include a processing module to complete the image processing steps, e.g., the accessory includes a communications module for communicating the acquired images to a remote device for image processing. Here, the communications module may be any suitable wireless communications module such as a Bluetooth, Wi-Fi, IRDA, NFC module or other short- or medium-range communications module. A switch 180 may be provided on the housing of the accessory, to be manually operated, and to control the controller. For instance, the switch 180 may be used to control the controller to establish a connection through the communications module 170 with the remote device. The switch may also control the controller to obtain through the image sensor the pre-injection or post-injection image. It will be appreciated that where the switch has alternative functions, the switch may be the same switch or there may be a plurality of switches each with their own function. Moreover, one or more of the switches may be automatically operated. For instance, a switch may be arranged to detect the placement or removal of the cap from the injection device, or the dose dispensing of the dose dispensing mechanism, or the operation of the dose setting mechanism. Here, the switches may also be provided on the injection device where appropriate or necessary and the attachment portion and receiving portions may also provide electrical contacts to electrically connect the two parts.

A power supply 190 is provided in the accessory to provide power to the respective parts.

Referring to FIG. 11, a method of managing a dosage regime is shown. The method comprises, at step S100, obtaining a pre-injection image. The pre-injection image records a position of the LDN 260 before an injection step is completed at step S200. The obtaining the pre-injection image may be triggered by operation of a switch 180, or alternatively, a post-injection image of a previous injection may be used or recalled from memory.

Step S200 comprises completing an injection step. The injection step includes dialing in a dose to be injected using the dose setting mechanism and activating the dose dispensing mechanism to dispense the medicament.

At step S300 a post-injection image is obtained. Again, the post-injection method is obtained by the image sensor 130 and the controller may be triggered to obtain the image by operation of a switch.

At step S400, the post-injection image and optionally the pre-injection image are transmitted to a remote device for image processing. The remote device processes the images and calculates the dose dialed measurement in image processing step S500. Here the dose dialed measurement may be a calculation including the displacement of the LDN 260 and other parameters of the dose dispensing mechanism such as screw pitch and diameter. After the image processing step calculates the dose dialed measurement, the remote device may transmit calculated measurement back to the communications module to be displayed by the display 140.

Alternatively, step S400 may be omitted and S500 may be performed by the accessory. In these embodiments, the calculated dose may or may not be transmitted to another device.

The user may use the calculated measurement displayed on the display 140 in a manual log book. However, in exemplary embodiments, the remote device includes a software application program to electronically log the calculated dose dialed measurement and optionally other injection parameters. For instance, the dosage management is implemented through a computer or the like. For instance, as an application on a smartphone or tablet or the like wherein the application monitors and alerts the user to the time and dosage of the injections. The application can also be used to input and record the injection parameters, for instance, to automatically log the time and date of the injection.

According to the above, the accessory 100 can be attached to an injection device 200 and in doing so, the optical sensor arrangement 130 in the accessory is aligned with the window 270 in the injection device. This may include causing an attachment portion 110 to engage a receiving portion and optionally for a part on the accessory to key with a part of the injection device. An injection is completed by the injection device 200 and the accessory 100 controlled to optically monitor a displacement of the LDN 260. The pre-injection and post-injection images are processed to calculate a dose dialed measurement, which is used in the dose management regime. By electronically logging or displaying the measurement for electronic logging, the dose management regime can be improved, for instance, by improving the recording accuracy or automation of the dose dialed measurement.

The accessory has been described herein as being attachable and detachable to an injection device. This is particularly useful for a disposable injection device wherein the resources of, for instance, the optical sensor arrangement can be reused between multiple injection devices. However, it is also envisaged that the accessory may be formed as an integral part of an injection device. In some examples, the injection device is a reusable injection device. When formed integrally, the accessory may be arranged to be permanently attached to the injection device or, for instance, the body of the accessory may be a common body of the injection device.

Although claims have been formulated in this application to particular combinations of features, it should be understood that the scope of the disclosure also includes any novel features or any novel combinations of features disclosed herein either explicitly or implicitly or any generalisation thereof, whether or not it relates to the same disclosure as presently claimed in any claim and whether or not it mitigates any or all of the same technical problems as does the present disclosure. The applicant hereby gives notice that new claims may be formulated to such features and/or combinations of features during the prosecution of the present application or of any further application derived therefrom.

Although several embodiments have been shown and described, it would be appreciated by those skilled in the art that changes may be made in these embodiments without departing from the principles of the present disclosure, the scope of which is defined in the claims.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codeable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An examples of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope of the present disclosure, which encompass such modifications and any and all equivalents thereof.

1-15. (canceled)

16. An apparatus comprising:
an accessory; and
an injection device wherein:
the accessory has an attachment portion configured to attach the accessory to the injection device, and a body that houses an optical sensor arrangement;
the injection device comprises a cartridge assembly and a housing, the housing contains a dose dispensing mechanism and a dose setting mechanism, the dose dispensing mechanism comprises a part that moves in response to movement of the dose setting mechanism and indicates a dose dialed by the dose setting mechanism, and the housing comprises a window aligned with the part of the dose dispensing mechanism,
wherein the attachment portion is configured to align the optical sensor arrangement to the window in the injection device to allow the optical sensor arrangement to detect a location of the part that moves in response to movement of the dose setting mechanism to determine the dose dialed by the dose setting mechanism.

17. The apparatus of claim 16, wherein the optical sensor arrangement comprises a sensor array configured to capture an image of the part.

18. The apparatus of claim 17, wherein the sensor array is a one-dimensional array.

19. The apparatus of claim 17, wherein the accessory is configured to cause the sensor array to capture a first image of the part that moves in response to movement of the dose setting mechanism after it has been detected that an injection has been completed.

20. The apparatus of claim 19, wherein the accessory is further configured to cause the sensor array to capture a second image of the part that moves in response to movement of the dose setting mechanism prior to an injection having started.

21. The apparatus of claim 20, wherein the accessory is configured to use both the first and second images to calculate a displacement of the part.

22. The apparatus of claim 21, wherein the accessory is configured to calculate the displacement of the part by detecting locations of edges in the first and second images and by comparing the positions of the edges between the first and second images.

23. The apparatus of claim 17, wherein the accessory comprises a communication module and is configured to use the communication module to communicate images acquired by the optical sensor arrangement to an external device for processing.

24. The apparatus of claim 23, wherein the communications module is a wireless communications module.

25. The apparatus of claim 16, wherein the part is a last dose nut threaded to a drive member of the dose setting mechanism, the last dose nut being arranged to advance along the drive member through a threaded engagement and in response to operation of the dose setting mechanism.

26. The apparatus of claim 16, wherein the injection device includes a receiving portion for receiving the attachment portion, and wherein the receiving portion is not covered by a cap of the injection device when the cap is operatively installed.

27. An accessory comprising:
an attachment portion configured to attach the accessory to an injection device having a dose setting mechanism; and
a body that houses an optical sensor arrangement,
wherein the attachment portion is configured to align the optical sensor arrangement to a window in the injection device, the window being aligned with a part that moves in response to movement of the dose setting mechanism to determine a dose dialed by the dose setting mechanism.

28. The accessory of claim 27, further comprising:
a communication module for communicating images acquired by the optical sensor arrangement to a remote device for image processing.

29. The accessory of claim 27, wherein the optical sensor arrangement is configured to capture a first image of the part that moves in response to movement of the dose setting mechanism after it has been detected that an injection has been completed.

30. The accessory of claim 29, wherein the optical sensor arrangement is further configured to capture a second image of the part that moves in response to movement of the dose setting mechanism prior to an injection having started.

31. The accessory of claim 30, further comprising:
a processor configured to use both the first and second images to calculate a displacement of the part.

32. The accessory of claim 31, wherein the processor is configured to calculate the displacement of the part by detecting locations of edges in the first and second images and by comparing the positions of the edges between the first and second images.

33. An injection device comprising:
a cartridge assembly; and
a housing containing a dose dispensing mechanism and a dose setting mechanism,
wherein the dose dispensing mechanism includes a last dose nut that moves in response to movement of the dose setting mechanism and indicates a dose dialed by the dose setting mechanism, and
the housing includes a window aligned with the last dose nut.

34. The injection device of claim 33, further comprising:
a cap that, when operatively connected to the injection device, covers part of the cartridge assembly, and
a receiving portion for receiving an attachment portion of an accessory, the receiving portion being uncovered by the cap when the cap is operatively connected.

35. The injection device of claim 33, wherein the cartridge assembly comprises a cartridge containing medicament.
